(19) United States
(12) Patent Application Publication
(10) Pub. No.: US 2023/0215569 A1
(43) Pub. Date: Jul. 6, 2023

(54) SYSTEMS AND METHODS FOR DETECTING DISEASES BASED ON THE PRESENCE OF VOLATILE ORGANIC COMPOUNDS IN THE BREATH

(71) Applicant: Opteev Technologies, Inc., Baltimore, MD (US)

(72) Inventors: Conrad Bessemer, Millersville, MD (US); Susan Bessemer, Millersville, MD (US); Biplab Pal, Ellicot City, MD (US)

(73) Assignee: Opteev Technologies, Inc., Baltimore, MD (US)

(21) Appl. No.: 18/150,119

(22) Filed: Jan. 4, 2023

Related U.S. Application Data
(60) Provisional application No. 63/296,187, filed on Jan. 4, 2022.

(51) Int. Cl. G16H 50/20; G01N 33/497

(52) U.S. Cl. CPC G16H 50/20; G01N 33/497; G01N 2033/4975

(57) ABSTRACT

Systems and methods are provided for detecting potentially fatal, and non-fatal diseases in an non-invasive, low-cost, and reliable manner by detecting the trace presence of volatile organic compounds (VOCs) in the human breath. The systems and methods can be home-based, non-invasive systems and methods for diagnosing CLD (chronic liver disease), CKD (chronic kidney disease), and other diseases using lifestyle-based, repetitive detection of VOCs in the human breath and an adaptive machine learning algorithm.

### BACKGROUND

Breath analysis is a non-invasive method for diagnosing illnesses by distinguishing, detecting, and quantifying endogenous volatile organic compound (VOC) concentrations. While VOCs also are released from urine, sputum, and feces, and can be detected from those sources in a non-invasive manner, exhaled breath is considered the metabolic by-product that can be handled the most easily, and hence has a clear advantage over the other types of by-products (see B. T. Larssonetal., “Gas Chromatography of Organic Volatiles in Human Breath and Saliva,” Actachem.scand, vol. 19, no. 1, pp. 159-164, 1965).

VOCs in the human breath come from blood that flows inside the alveoli of the lungs and comes into contact with the alveolar breath. The VOC exchange across the capillary membranes of the alveoli is affected by the composition of the blood. As a result, VOCs in the expelled breath come from the blood flowing in the body's headspace (see V. Richter and J. Tonzetich, “The Application of Instrumental Technique for the Evaluation of Odoriferous Volatiles from Saliva and Breath,” Archives of Oral Biology, vol. 9, no. 1, pp. 47-53, 1964).

Earlier studies show a correlation between VOCs in the human breath and diseases like pneumonia, pulmonary tuberculosis (TB), asthma, lung cancer, liver diseases, kidney diseases, etc. (see R. Teranishi, T. Mon, A. B. Robinson, P. Cary, and L. Pauling, “Gas Chromatography of Volatiles from Breath and Urine,” Analytical Chemistry, vol. 44, no. 1, pp. 18-20, 1972); and such VOCs can be identified by the smell of the breath (see European Patent No. EP2459984B1).

Analytical chemical methods like gas chromatography-mass spectrometry (GC-MS), proton transfer reaction mass spectrometry (PTR-MSO), and ion mobility spectrometry (IMS) can be used to identify the vast number of VOCs that are emitted in the exhaled breath of a human. Some VOCs are in the order of parts-per-million (ppm) or parts-per-billion (ppb) (see A. Manolis, “The Diagnostic Potential of Breath Analysis,” Clinical Chemistry, vol. 29, no. 1, pp. 5-15, 1983). For example, an earlier study utilizing GC-MS proved the presence of over one thousand VOCs in the exhaled breath of several human participants (see M. Phillips, M. Sabas, and J. Greenberg, “Increased Pentane and Carbon Disulfide in the Breath of Patients with Schizophrenia,” Journal of Clinical Pathology, vol. 46, no. 9, pp. 861-864, 1993).

According to Fink et al., IMS was utilized to identify 42 distinct analytes with great precision (see D. Smith, Breath Analysis For Clinical Diagnosis & Therapeutic Monitoring (With Cd-rom), World Scientific, 2005).

The above-noted analytical approaches, however, can be bulky, expensive, and complex, and require qualified operators. These approaches, therefore, can have limited utility in home-based self practice due to high cost and user logistics, and thus may be unsuitable for personal healthcare, particularly when patients need personal, portable machines for continuous monitoring at home (see C. Davis and J. Beauchamp, Volatile Biomarkers: Non-Invasive Diagnosis in Physiology and Medicine, Newnes, 2013). For at least these reasons, developing low-cost, portable, and dependable breath analysis equipment for clinical diagnoses and monitoring is desirable.

### SUMMARY

In aspect of the disclosed technology, a VOC detection bio-sensor, which is on a silk substrate functionalized with PPY (polypyrrole), RGO (reduced graphene oxide) or CNT (carbon nanotube), or an organic transistor whose gate has been functionalized with RGO reduced with curcumin or similar reducing agent, is provided. These biosensors are low cost, highly sensitive, and easy to produce in large quantities. However, the response characteristics of the biosensors are not reproducible in a batch, and thus they are unsuitable for mass scale calibration and need regular individual calibration which makes it very difficult to use for any quantitative diagnostics. These issues are addressed by the present technology using adaptive AIML algorithms and reference gases like carbon dioxide or water vapor whose ppm level remains same in exhaled breadth in every human being.

In another aspect, the disclosed technology is directed to the early detection of potentially fatal, and non-fatal, diseases in a non-invasive, low-cost, and reliable manner by detecting the trace presence of VOCs in the human breath. The use of this technology can help home-based and other users to obtain a diagnosis and a predictive prognosis of CLD, CKD, lung cancer, asthma, diabetes, and other diseases. In one non-limiting embodiment, a system optically detects the presence of acetone and ammonia in the human breadth in the range of, for example, about 10 ppb to about 5,000 ppb. This information can be obtained at different points in the human metabolic cycle, such as during fasting, after dinner, etc.

The medical community has long recognized that humans exhale VOCs, such as ketones, aldehydes, and alcohol, as a result of human metabolic activity. But the concentration of some of the VOCs in the breath changes when an individual is affected by diseases such as the above-mentioned diseases. The disclosed technology can be used to detect diseases based on the presence of a particular VOC, or biomarker, in the exhaled breath.

In another aspect of the disclosed technology, an optical or other type of sensor is used to sense the presence, and level of the VOC. The output of the sensor is analysed to detect the disease, and the dispersion of the acquired data can be reduced by additional lifestyle and clinical data to increase the reliability of criteria for distinguishing between healthy and unhealthy, i.e., diseased, patients.

In another aspect to the disclosed technology, an edge intelligent device configured to track bio-markers of diseases by measuring VOCs of the patient on different metabolic conditions (after and before fasting & eating etc.) and by building a heterogenous predictive model by combining the data obtained from the VOC levels after specified metabolic conditions with personal life-style information of the patients to reduce the dispersion of the bio-marker data for the purpose of clean and reliable separation between healthy and un-healthy patients, and a home-based platform for early detection of critical diseases like CKD, CLD, etc.

In another aspect to the disclosed technology, the device is further configured to receive dynamic voltage-current signal from a VOC detecting bio-sensor which is made out of silk substrate functionalized with PPY (Polypyrrole), RGO (reduced graphene oxide) or CNT (carbon nanotube); or an organic transistor whose gate has been functionalized with RGO reduced with Curcumin or similar reducing agent.

In another aspect to the disclosed technology, a method for creating a diagnostic tool for determining the presence or absence of a disease includes measuring levels of one or more volatile organic compounds in the breath of one or more individuals known to be afflicted with the disease, at a predetermined point in a metabolic cycle of the one of more individuals known to be afflicted with the disease; obtaining lifestyle data about the one or more individuals known to be afflicted with the disease; and measuring levels of the one or more volatile organic compounds in the breath of one or more individuals known not to be afflicted with the disease at a predetermined point in a metabolic cycle of the one of more individuals known not to be afflicted with the disease.

The method further includes obtaining lifestyle data about the one or more individuals known not to be afflicted with the disease; assembling a data set comprising: the levels of the one or more volatile organic compounds in the breath of one or more individuals known to be afflicted with the disease; the lifestyle data about the one or more individuals known to be afflicted with the disease; the levels of one or more volatile organic compounds in the breath of one or more individuals known not to be afflicted with the disease; and the lifestyle data about the one or more individuals known not to be afflicted with the disease; reducing a dimensionality of a data set; creating a classification model for determining the presence and absence of the disease based on the reduced-dimensionality data set; and validating the classification model.

In another aspect to the disclosed technology, a method for determining the presence or absence of a disease in an individual includes measuring levels of one or more volatile organic compounds in the breath of the individual, at a predetermined point in a metabolic cycle of the individual; obtaining lifestyle data about the individual; inputting the levels of one or more volatile organic compounds in the breath of the individual and the lifestyle data about the individual into a classification model, and using the classification model to determine the presence or absence of the disease in the individual.

In another aspect to the disclosed technology, measuring levels of one or more volatile organic compounds in the breath of one or more individuals known to be afflicted with the disease comprises measuring the levels of one or more volatile organic compounds in the breath of the one or more individuals known to be afflicted with the disease using a VOC detecting bio-sensor comprising silk substrate functionalized with PPY (Polypyrrole), RGO (reduced graphene oxide) or CNT (carbon nanotube); or an organic transistor comprising a gate functionalized with RGO reduced with curcumin or a similar reducing agent.

In another aspect to the disclosed technology, measuring levels of the one or more volatile organic compounds in the breath of one or more individuals known not to be afflicted with the disease includes measuring the levels of the one or more volatile organic compounds in the breath of the one or more individuals known not to be afflicted with the disease using the VOC detecting bio-sensor.

In another aspect of the disclosed technology, a system method for creating a diagnostic tool for determining the presence or absence of a disease in an individual includes a computing device having a processor, a memory communicatively couped to the processor, and computer-executable instructions stored on the memory, wherein the processor, upon executing the computer-executable instructions, causes the computing device to: assemble a data set that includes the levels of one or more volatile organic compounds in the breath of one or more individuals known to be afflicted with a disease; lifestyle data about the one or more individuals known to be afflicted with the disease; the levels of one or more volatile organic compounds in the breath of one or more individuals known not to be afflicted with the disease; and the lifestyle data about the one or more individuals known not to be afflicted with the disease; reduce a dimensionality of a data set; create a classification model for determining the presence and absence of the disease based on the reduced-dimensionality data set; and validate the classification model.

### DESCRIPTION OF THE DRAWINGS

The following drawings are illustrative of particular embodiments of the present disclosure and do not limit the scope of the present disclosure. The drawings are not to scale and are intended for use in conjunction with the explanations provided herein. Embodiments of the present disclosure will hereinafter be described in conjunction with the appended drawings.

FIG. 1 is a table of levels of various VOCs in the breath samples of a non-alcoholic fatty liver disease group, and a group of healthy individuals.

FIG. 2 is a table of acetone concentration data in healthy individuals, and individuals with liver disease.

FIG. 3 is a table of data sampling parameters for building VOC profiles for individual patients.

FIG. 4. is a flow chart of a process for developing an adaptive model for separating diseased and healthy individuals using VOC profiles of the individuals.

### DETAILED DESCRIPTION

The inventive concepts are described with reference to the attached figures, wherein like reference numerals represent like parts and assemblies throughout the several views. The figures are not drawn to scale and are provided merely to illustrate the instant inventive concepts. The figures do not limit the scope of the present disclosure or the appended claims. Several aspects of the inventive concepts are described below with reference to example applications for illustration. It should be understood that numerous specific details, relationships, and methods are set forth to provide a full understanding of the inventive concepts. One having ordinary skill in the relevant art, however, will readily recognize that the inventive concepts can be practiced without one or more of the specific details or with other methods. In other instances, well-known structures or operation are not shown in detail to avoid obscuring the inventive concepts.

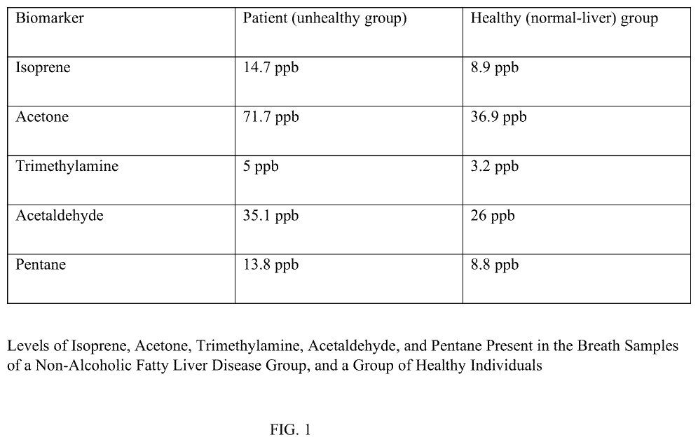

Many researchers have reported VOCs or biomarkers specific to particular diseases, but vary on the specific concentrations of VOCs present in the breath of diseased and healthy persons. For example, according to a research paper published by Naim Alkhouri (see https://doi.org/10.1097/meg.0b013e3283650669), isoprene, acetone, trimethylamine, acetaldehyde, and pentane at the levels noted in FIG. 1 are present in the breath samples of an NAFLD (non-alcoholic fatty liver disease) group, in contrast to the corresponding VOC levels found in the breath samples of a normal-liver group and listed in FIG. 1.

A journal article titled “GC-MS Analysis of Breath Oder Compounds in Liver Patients” (https://doi.org/10.1016/j.jchromb.2008.08.031) reports that dimethyl sulfide, acetone, 2-butanone, and 2-pentanone are present in increased levels in the breath of patients with liver disease. In contrast, the levels of indole and dimethyl selenide were decreased in such patients. A disease detecting model was built with a sensitivity and specificity of 100 percent and 70 percent, respectively, based on acquired data.

Another study titled “Isoprene in the Exhaled Breath is a Novel Biomarker for Advanced Fibrosis in Patients with Chronic Liver Disease: A Pilot Study” (10.1038/ctg.2015.40) reports that, of 61 patients, 33% had advanced fibrosis, 44% had chronic hepatitis C, 30% had non-alcoholic fatty liver disease, and 26% had other CLD. SIFT-MS analysis of exhaled breath revealed that patients with advanced fibrosis had significantly lower values of six compounds in comparison to patients without advanced fibrosis. Isoprene is an endogenous VOC that is a by-product of cholesterol biosynthesis.

Another study, titled “The Breath Prints in Patients with Liver Disease Identify Novel Breath Biomarkers in Alcoholic Hepatitis” (hllps://doi.org/10.1016/j.cgh.2013.08.048), reports increased levels of 2-propanol, acetaldehyde, acetone, ethanol, pentane, and trimethylamine [TMA] compounds in patients with liver disease in comparison to the levels in control subjects.

While the above studies each report a baseline for distinguishing healthy from unhealthy individuals, the baselines for particular VOCs in the breath are inconsistent for the same disease across the studies; and the baselines are so widely dispersed that separating healthy and unhealthy people is not possible merely by detecting the presence and/or level of the relevant biomarker.

Comparing the data from the above studies shows inconsistencies in the concentrations of the VOCs emitted by diseased and healthy individuals, invalidating the disease detection model built in some, or all of the studies. The following are at least some of the limitations seen when comparing the data across the studies.

The baseline values of different biomarkers reported by different studies were limited by sample data.

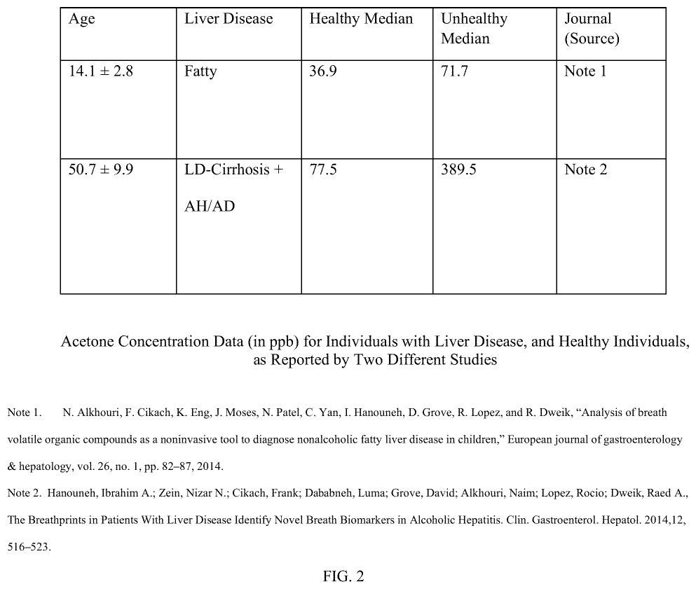

Different studies considered specific age groups and different clinical histories to determine baselines for different biomarkers. As a result, there is no consistent pattern found for both healthy and liver-disease data, and it is not possible to find a unique baseline for the biomarker (VOC) concentration level for each effective biomarker. For example, the acetone concentration data (in ppb) for liver disease reported by two different studies gives the data presented in FIG. 2.

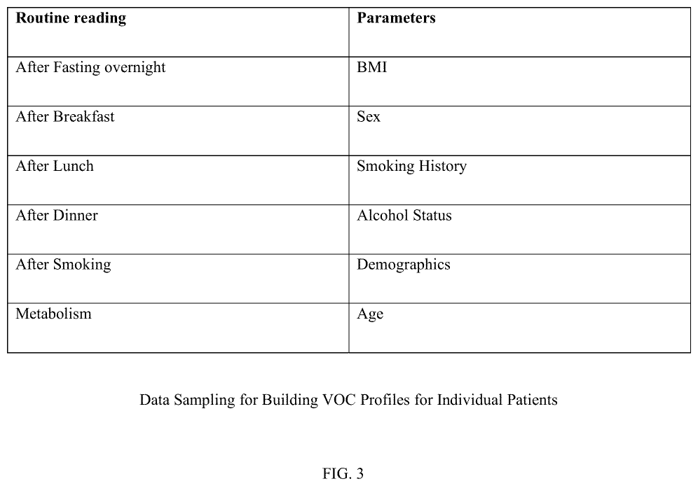

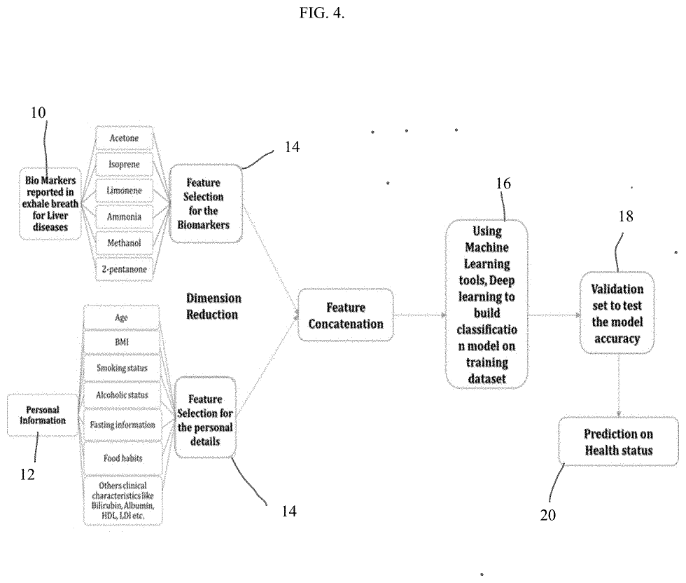

The disclosed technology builds an adaptive model for separating diseased and healthy individuals using a data science method/condition to reduce the dispersion of the baseline by building a VOC profile of each patient. The process for developing the model is depicted in FIG. 4. The VOC profile is established by routinely taking readings of the VOC levels in the breath of diseased and healthy individuals at certain points in the metabolic cycle; and acquiring and considering the parameters noted in FIG. 3 relating to the individuals.

By taking VOC readings from an individual over one or two days, the VOC profile of the individual can be built, and can be used to identify diseased vs. healthy individuals.

The VOC readings can be obtained, for example, by a VOC detecting bio-sensor that is made of silk substrate functionalized with PPY (Polypyrrole), RGO (reduced graphene oxide), or CNT (Carbon Nanotube), or an organic transistor whose gate has been functionalized with RGO reduced with curcumin or similar reducing agent. The output of the sensor can be fed to an edge device or other computing device as a dynamic voltage-current signal.

Summary Analytical Approach

Various studies have reported that specific VOCs or biomarkers in the human breath can provide significant clues to detecting the health of the liver and kidney of an individual. Thus, these markers can be used as an indicator for the early detection of CLD and CKD. Several studies, however, show different optimal levels for the concentration of the biomarker (in ppb) used to detect the disease. With the variation in the range of the samples studied in the existing literature, it is not believed to be possible to demarcate a single baseline for a particular biomarker to identify the presence of the disease. The studies also reveal that the variation of the CLD can be of different forms like mild cirrhosis, cirrhosis with AH, fibrosis, and advanced fibrosis. The presence of these diseases is identified from the concentration (in ppb) of the biomarkers in the breath emitted during exhalation. In addition to the biomarkers, knowledge of the age, smoking status, alcohol-use status, BMI, fasting status, food habits, etc. of the patient can help provide a better understanding of the health of a person's liver and kidneys.

Data Acquisition

After a fast of four to eight hours, exhaled breath samples are collected from people aged ten to 70 who have different types of liver or kidney diseases, and healthy individuals (activity 10 of FIG. 4). Lifestyle data also is collected (activity 12 of FIG. 4). Data related to different significant biomarkers is collected from samples of exhaled breath. Clinical variables such as body mass index (BMI), diabetic status, etc., and lifestyle information such as smoking status, alcohol-use status, etc. are recorded from each healthy and unhealthy (CKD, CLD) subject.

Features Reduction

A particular dataset may contain many input features, making the predictive modelling task more complicated for that dataset. Because it is complicated to visualize or make predictions for a training dataset with a high number of features, dimensionality reduction techniques are required for such cases. If a machine learning model is trained on high-dimensional data, it becomes overfitted, resulting in poor performance. The dimensionality reduction technique is a way of converting a higher-dimension dataset into a lesser-dimension dataset, ensuring that it provides similar information. Principal Component Analysis, Forward Feature Selection, Backward Feature Selection are data reduction techniques that can be used before the model development, to reduce the dimensionality of the dataset acquired as described above (activity 14 of FIG. 4).

Classification Model

A classification for predicting healthy and unhealthy subjects can be built on the dimension-reduced data set using a parametric classification model like Logistic Regression, a machine learning model like Decision Tree, Random Forest, or a deep learning model like Neural Network (activity 16 of FIG. 4). The best model subsequently can be selected based on a cross-validation score (activity 18 of FIG. 4).

The resulting adaptive model for separating diseased and healthy individuals based on VOCs in the breath subsequently can be used to determine or predict the health status of individuals (activity 20 in FIG. 4).

The above-noted steps of dimensionality reduction, feature concatenations, classification, validation, and prediction of health status can be made by a suitable computing device, such as but not limited to an edge-cloud server, programmed with computer-executable instructions that, when executed by the computing device, cause the computing device to carry out the logical operations in accordance with the above-noted techniques.

We claim:

1. An edge intelligent device configured to track bio-markers of diseases by measuring VOCs of the patient on different metabolic conditions (after and before fasting & eating etc.) and by building a heterogenous predictive model by combining the data obtained from the VOC levels after specified metabolic conditions with personal life-style information of the patients to reduce the dispersion of the bio-marker data for the purpose of clean and reliable separation between healthy and un-healthy patients, and a home-based platform for early detection of critical diseases like CKD, CLD, etc.

2. The edge intelligent device of claim 1, wherein the device is further configured to receive dynamic voltage-current signal from a VOC detecting bio-sensor which is made out of silk substrate functionalized with PPY (Polypyrrole), RGO (reduced graphene oxide) or CNT (carbon nanotube) or an organic transistor whose gate has been functionalized with RGO reduced with Curcumin or similar reducing agent.

3. A method for creating a diagnostic tool for determining the presence or absence of a disease, comprising:
measuring levels of one or more volatile organic compounds in the breath of one or more individuals known to be afflicted with the disease, at a predetermined point in a metabolic cycle of the one of more individuals known to be afflicted with the disease;
obtaining lifestyle data about the one or more individuals known to be afflicted with the disease;
measuring levels of the one or more volatile organic compounds in the breath of one or more individuals known not to be afflicted with the disease at a predetermined point in a metabolic cycle of the one of more individuals known not to be afflicted with the disease;
obtaining lifestyle data about the one or more individuals known not to be afflicted with the disease;
assembling a data set comprising: the levels of the one or more volatile organic compounds in the breath of one or more individuals known to be afflicted with the disease; the lifestyle data about the one or more individuals known to be afflicted with the disease; the levels of one or more volatile organic compounds in the breath of one or more individuals known not to be afflicted with the disease; and the lifestyle data about the one or more individuals known not to be afflicted with the disease;
reducing a dimensionality of a data set;
creating a classification model for determining the presence and absence of the disease based on the reduced-dimensionality data set; and
validating the classification model.

4. A method for determining the presence or absence of a disease in an individual, comprising:
measuring levels of one or more volatile organic compounds in the breath of the individual, at a predetermined point in a metabolic cycle of the individual;
obtaining lifestyle data about the individual;
inputting the levels of one or more volatile organic compounds in the breath of the individual and the lifestyle data about the individual into the classification model of claim 3; and
using the classification model to determine the presence or absence of the disease in the individual.

5. The method of claim 3, wherein measuring levels of one or more volatile organic compounds in the breath of one or more individuals known to be afflicted with the disease comprises measuring the levels of one or more volatile organic compounds in the breath of the one or more individuals known to be afflicted with the disease using a VOC detecting bio-sensor comprising silk substrate functionalized with PPY (Polypyrrole), RGO (reduced graphene oxide) or CNT (carbon nanotube); or an organic transistor comprising a gate functionalized with RGO reduced with curcumin or a similar reducing agent.

6. The method of claim 5, wherein measuring levels of the one or more volatile organic compounds in the breath of one or more individuals known not to be afflicted with the disease comprises measuring the levels of the one or more volatile organic compounds in the breath of the one or more individuals known not to be afflicted with the disease using the VOC detecting bio-sensor.
